(19) United States
(12) Patent Application Publication
(10) Pub. No.: US 2025/0345091 A1
(43) Pub. Date: Nov. 13, 2025

(54) FLEXIBLE MEMBER TO MAINTAIN PATENCY OF DELIVERY DEVICE

(71) Applicant: Boston Scientific Scimed, Inc., Maple Grove, MN (US)

(72) Inventors: Lauren Koon, Mississauga (CA); Christian Balkovec, Cambridge (CA)

(21) Appl. No.: 19/202,785

(22) Filed: May 8, 2025

Related U.S. Application Data
(60) Provisional application No. 63/645,296, filed on May 10, 2024.

(51) Int. Cl. A61B 17/34; A61B 18/14

(52) U.S. Cl. CPC A61B 17/3468; A61B 18/1487

(57) ABSTRACT

A system for perforating a tissue within a heart is disclosed. The system includes a delivery device having an elongated body including a body length, a proximal end and a distal end portion terminating in a distal tip. The elongated body defines a delivery lumen extending through the elongate body and dimensioned to slidingly receive at least one or more wires. The system further includes a secondary device having a proximal end and a distal end portion terminating in a distal tip dimensioned to extend longitudinally within the delivery lumen of the delivery device towards the distal tip of the device. The system further includes a shapeable pre-formed member longitudinally advanced through the elongated body of the delivery device. The shapeable pre-formed member does not extend longitudinally within the delivery lumen of the delivery device, and the pre-formed member maintains lumen patency of the delivery device.

### CROSS REFERENCE TO RELATED APPLICATIONS

This application claims priority to U.S. Provisional Patent Application No. 63/645,296 entitled “FLEXIBLE MEMBER TO MAINTAIN PATENCY OF DELIVERY DEVICE,” filed May 10, 2024, which is hereby incorporated by reference in its entirety.

### TECHNICAL FIELD

The present disclosure relates to medical systems and methods for accessing a tissue within a heart of a patient. More specifically, the present disclosure relates to a delivery device comprising a shapeable pre-formed member which provides flexibility and patency to the delivery device.

### BACKGROUND

In general, commercially available devices for accessing and perforating a tissue within a heart of a patient include a hollow lumen used to cannulate one or more other medical devices, for example, a perforation device, longitudinally through the hollow lumen. A problem with medical devices having a hollow lumen, such as a catheter or a dilator, is that these devices are susceptible to kinking, loss in lumen patency or reduction in lumen inner diameter when the device travels within the body and into the heart of the patient. Medical device kinking refers to the bending or folding of the device, which can lead to a loss in lumen patency. Lumen patency is crucial for devices in accessing and perforating a tissue within a heart of a patient as it ensures the unobstructed flow of fluids or other devices through the hollow lumen. Kinking and loss of lumen patency may affect device integrity during use, and, in some instances, the hollow lumen may no longer be able to be used to cannulate one or more other medical devices therein. Thus, when a medical device with a hollow lumen kinks, it may compromise the device's functionality and effectiveness. The kinking may result in a reduction or complete blockage of the lumen, hindering the intended purpose of the device.

Against this background, there exists a continuing need in the industry to provide improved devices and methods for gaining access to and perforating a tissue within a patient's heart. An object of the present invention is therefore to provide such an apparatus.

### SUMMARY

In Example 1, a system for perforating a tissue within a heart includes a delivery device comprising an elongated body having a body length, a proximal end and a distal end portion terminating in a distal tip, the elongated body defining a delivery lumen extending through the elongate body and dimensioned to slidingly receive at least one or more wires. The system also includes a secondary device having a proximal end and a distal end portion terminating in a distal tip dimensioned to extend longitudinally within the delivery lumen of the delivery device towards the distal tip of the device. The system further includes a shapeable pre-formed member longitudinally advanced through the elongated body of the delivery device; wherein the shapeable pre-formed member does not extend longitudinally within the delivery lumen of the delivery device; and wherein the pre-formed member maintains lumen patency of the delivery device.

Example 2 is the system of Example 1 wherein the delivery device further includes at least one support lumens, and wherein the at least one support lumens is adjacent the delivery lumen.

Example 3 is the system of Example 2 wherein the pre-formed member is longitudinally advanced through the at least one support lumens.

Example 4 is the system of Example 1 wherein the elongated body of the delivery device further includes a device wall adjacent to the delivery lumen, and wherein the pre-formed member is longitudinally advanced through a portion of the wall of the elongated body.

Example 5 is the system of Example 4 wherein the pre-formed member is embedded into the wall of the delivery device.

Example 6 is the system of Example 1 wherein the pre-formed member allows flexibility to the device while minimizing kinking of the elongated body of the device.

Example 7 is the system of Example 1 wherein the pre-formed member is conformed into a desired shape by a user.

Example 8 is the system of Example 7 wherein the pre-formed member is longitudinally advanced through the at least one support lumens of the delivery device to achieve a specific device shape as the delivery device is advancing within the heart.

Example 9 is the system of Example 1 wherein the delivery device is a dilator comprising an elongated body having a body length, a proximal end and a distal end portion terminating in a distal tip, the elongated body defining a delivery lumen extending through the elongate body and dimensioned to slidingly receive at least one or more wires.

Example 10 is the system of Example 9 wherein the dilator includes at least one support lumens, and wherein the pre-formed member is longitudinally advanced through the at least one support lumens of the dilator.

Example 11 is the system of Example 1 wherein the delivery device is an elongated catheter comprising an elongated body having a body length, a proximal end and a distal end portion terminating in a distal tip, the elongated body defining a delivery lumen extending through the elongate body and dimensioned to slidingly receive at least one or more devices, and wherein the elongated catheter is adapted to receive and allow longitudinal translation of the pre-formed member therein.

Example 12 is the system of Example 1 wherein the pre-formed member is solid, and wherein the pre-formed member does not define a lumen.

Example 13 is the system of Example 1 wherein the pre-formed member is selected from the group consisting of stainless steel and nitinol.

Example 14 is the system of Example 1 wherein the pre-formed member is longitudinally advanced through the entire elongated body of the delivery device.

Example 15 is the system of Example 1 wherein the pre-formed member is longitudinally advanced through a portion of the elongated body of the delivery device.

In Example 16, a system for perforating a tissue within a heart includes a delivery device comprising an elongated body having a body length, a proximal end and a distal end portion terminating in a distal tip, the elongated body defining a delivery lumen extending through the elongate body and dimensioned to slidingly receive at least one or more wires. The system also includes a secondary device having a proximal end and a distal end portion terminating in a distal tip dimensioned to extend longitudinally within the delivery lumen of the delivery device towards the distal tip of the device. The system further includes a shapeable pre-formed member longitudinally advanced through the elongated body of the delivery device; wherein the shapeable pre-formed member does not extend longitudinally within the delivery lumen of the delivery device; and wherein the pre-formed member maintains lumen patency of the delivery device.

Example 17 is the system of Example 16 wherein the delivery device further includes at least one support lumens, wherein the at least one support lumens is adjacent the delivery lumen, and wherein the pre-formed member is longitudinally advanced through the at least one support lumens.

Example 18 is the system of Example 16 wherein the elongated body of the delivery device further includes a device wall adjacent to the delivery lumen, and wherein the pre-formed member is longitudinally advanced through a portion of the wall of the elongated body.

Example 19 is the system of Example 18 wherein the pre-formed member is embedded into the wall of the delivery device.

Example 20 is the system of Example 16 wherein the pre-formed member allows flexibility to the device while minimizing kinking of the elongated body of the device.

Example 21 is the system of Example 16 wherein the pre-formed member is conformed into a desired shape by a user.

Example 22 is the system of Example 21 wherein the pre-formed member is longitudinally advanced through the at least one support lumens of the delivery device to achieve a specific device shape as the delivery device is advancing within the heart.

Example 23 is the system of Example 16 wherein the delivery device is a dilator comprising at least one support lumens, and wherein the pre-formed member is longitudinally advanced through the at least one support lumens of the dilator.

Example 24 is the system of Example 16 wherein the delivery device is an elongated catheter, and wherein the elongated catheter is adapted to receive and allow longitudinal translation of the pre-formed member therein.

Example 25 is the system of Example 16 wherein the pre-formed member is solid, and wherein the pre-formed member does not define a lumen.

Example 26 is the system of Example 16 wherein the pre-formed member is selected from the group consisting of stainless steel and nitinol.

In Example 27, a system for perforating a tissue within a heart includes a delivery device comprising an elongated body having a body length, a proximal end and a distal end portion terminating in a distal tip, the elongated body defining a delivery lumen and at least one support lumens extending through the elongate body and dimensioned to slidingly receive at least one or more wires. The system also includes a puncture device having a proximal end and a distal end portion terminating in a distal tip dimensioned to extend longitudinally within the delivery lumen of the delivery device towards the distal tip of the device. The system further includes a shapeable pre-formed member longitudinally advanced through the at least one support lumens of the delivery device; wherein the pre-formed member maintains lumen patency of the delivery device.

Example 28 is the system of Example 27 wherein the pre-formed member is longitudinally advanced through the at least one support lumens of the delivery device to achieve a specific device shape as the delivery device is advancing within the heart.

Example 29 is the system of Example 27 wherein the delivery device is a dilator comprising at least one support lumens, and wherein the pre-formed member is longitudinally advanced through the at least one support lumens of the dilator.

Example 30 is the system of Example 27 wherein the delivery device is an elongated catheter, and wherein the elongated catheter is adapted to receive and allow longitudinal translation of the pre-formed member therein.

Example 31 is the system of Example 27 wherein the pre-formed member is solid, and wherein the pre-formed member does not define a lumen.

Example 32 is the system of Example 27 wherein the pre-formed member is selected from the group consisting of stainless steel and nitinol.

Example 33 is the system of Example 27 wherein the pre-formed member is longitudinally advanced through the entire elongated body of the delivery device.

Example 34 is the system of Example 27 wherein the pre-formed member is longitudinally advanced through a portion of the elongated body of the delivery device.

In Example 35 a method for perforating a tissue within a heart includes providing a delivery device comprising an elongated body having a body length, a proximal end and a distal end portion terminating in a distal tip, the elongated body defining a delivery lumen extending through the elongate body and dimensioned to slidingly receive at least one or more wires. The method for perforating a tissue within a heart also includes advancing a secondary device having a proximal end and a distal end portion terminating in a distal tip dimensioned to extend longitudinally within the delivery lumen of the delivery device towards the distal tip of the device. The method for perforating a tissue within a heart further includes advancing a shapeable pre-formed member longitudinally advanced through the elongated body of the delivery device; wherein the shapeable pre-formed member does not extend longitudinally within the delivery lumen of the delivery device; and wherein the pre-formed member maintains lumen patency of the delivery device.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1B are schematic illustrations of medical procedures within a patient's heart for gaining access to the transseptal and epicardial space, according to embodiments of the present disclosure.

FIG. 2 is an illustration an access system for perforating a tissue within a heart of a patient having a delivery device defining a delivery lumen, according to embodiments of the present disclosure.

FIGS. 3A-4 are schematic illustrations of the delivery device and a shapeable pre-formed member which provides flexibility and patency to the delivery device, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

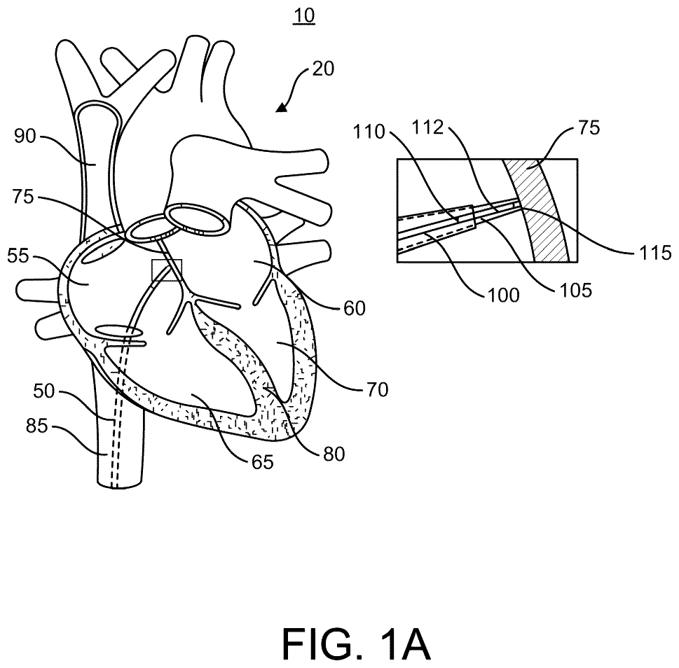

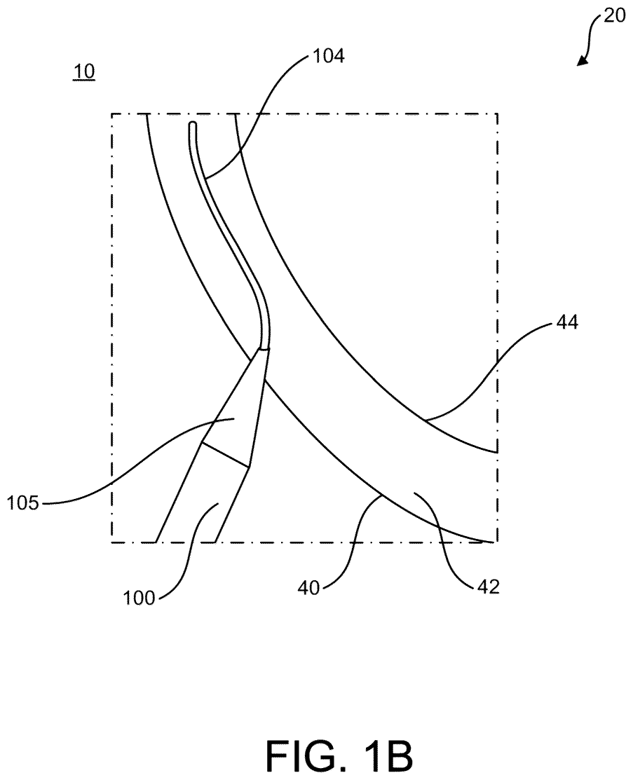

FIGS. 1A and 1B are schematic illustrations of a medical procedure within a patient's heart for gaining transseptal access as well as access to the epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device 110 having distal end portion 112 terminating in a tip electrode 115, and a guidewire. In various embodiments, the puncture device 110 is a mechanical puncture device (e.g., a needle) or an RF perforation device. The puncture device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20.

When the guidewire, sheath 100 and dilator 105 have been positioned in the SVC 90, the guidewire is removed from the body, and the sheath 100 and the dilator 105 are retracted so that their distal ends are positioned in the right atrium 55. In an embodiment, the puncture device 110 described can then be introduced into the dilator 105, and advanced toward the heart 20. In other embodiments, the puncture device 110 described may be introduced prior to the retraction of the sheath 100 and the dilator 105 from the SVC into the right atrium 55. The puncture device 110 is then positioned such that the tip electrode 115 is aligned with or protruding slightly from the distal end of the dilator 105. In embodiments where the puncture device 110 is an RF perforation device, with the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least 200 V RMS, or in certain embodiments about 565 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 0.1 second and about 5 seconds. In other embodiments, the step of delivering energy occurs over a period of about 300 milliseconds.

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110, such as a needle (or other mechanical puncture device). Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. In an embodiment, the sheath 100 and the dilator 105 may then be withdrawn to leave a guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

In an embodiment, many medical procedures, including the above-mentioned medical procedures, for diagnosing or treating physiological ailments originating within the heart include medical devices having a hollow lumen. The hollow lumen of a medical device refers to the interior space, internal conduit or channel within a medical device designed to facilitate the precise and controlled delivery of substances, one or more wires and/or devices within the cardiovascular system—such as the passage of fluids, gases, medical devices or other substances. In an embodiment, the devices having a hollow lumen may include a catheter, an introducer sheath, a dilator, a guidewire, among others. In an embodiment, the hollow lumen of the devices may vary in size, shape, and material composition based on the intended application. In an embodiment, for example, a catheter for the above-mentioned medical procedures for diagnosing or treating physiological ailments originating within the heart may include at least one or more hollow lumen serving various purposes, such as draining fluids, delivering medications, performing diagnostic procedures, or delivering other medical devices longitudinally within the catheter lumen. Another example of a device having a hollow lumen is a dilator, as will be discussed in greater detail below. The present disclosure describes novel systems and methods for providing safe access to the heart. As will be explained in greater detail herein, the embodiments of the present disclosure improve the functionality and effectiveness of medical devices having at least one or more hollow lumen.

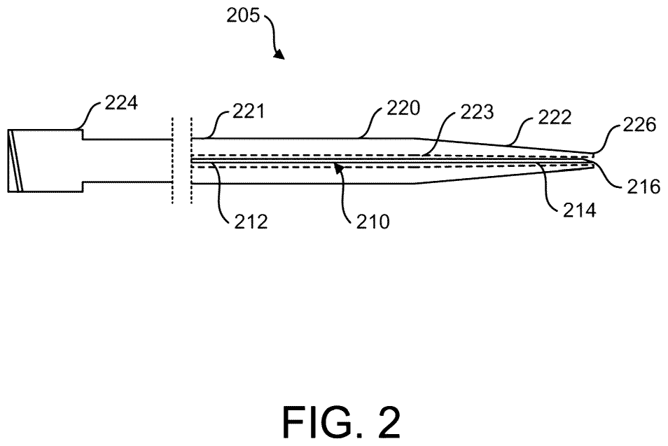

FIG. 2 is an illustration of an access system for perforating a tissue within a heart of a patient having a delivery device 205 defining a delivery lumen 223, according to an embodiment of the present disclosure. As shown, the delivery device 205 includes an elongated body 220 having a body length, a proximal end portion 221 and an opposite distal end portion 222 terminating in a distal tip 226. As shown, the delivery device 205 further includes the elongated body 220 defining a delivery lumen 223 extending through the elongate body 220 and dimensioned to slidingly receive at least one or more wires 210. In an embodiment, the one or more wires may include a secondary device 210. In an embodiment, the delivery device may be a dilator. In an embodiment, the delivery device may further include a handle 224 having a handle length, as shown in FIG. 2, with the delivery lumen 223 extending longitudinally through the handle 224 and the elongate body 220. In an embodiment, the handle 224 is connected to the proximal end portion 221 of the elongate body 220. In an embodiment, the body length and the handle length of the delivery device 205 together define a delivery device length. Additionally, as shown, the delivery lumen 223 of the delivery device 205 is dimensioned to slidingly receive the secondary device 210. In an embodiment, the delivery device may be a dilator, a catheter, an introducer sheath, a guidewire, or any other medical delivery devices defining at least one or more hollow lumen within.

As can be further seen from FIG. 2, the secondary device 210 includes a device body having a proximal portion 212 and a distal portion 214 extending from the proximal portion 212 and terminating in a distal tip 216. As shown, the secondary device 210 is dimensioned to extend longitudinally within the delivery lumen 223 of the delivery device 210 towards the distal tip 226 of the device 210. In an embodiment, the secondary device 210 may include a perforation device, a guidewire, or any other devices that may extend longitudinally through a hollow lumen. In some embodiments, a perforation device 210 may be an RF perforation device. In certain embodiments, the distal tip 216 may include a distal tip electrode (e.g., a tip electrode such as described above in connection with FIGS. 1A-1B). In other embodiments, the perforation device 210 may be a mechanical perforation device. As will be appreciated, in some embodiments, the length of the secondary device 210 is greater than the length of the delivery device 205 so that part of the proximal portion 212 of the perforation device 210 extends proximally of the handle 224 (not shown) when the distal portion 214, particularly the distal tip 216, extends distally of the device 205, thus allowing the proximal portion 212 to be manipulated by the user as needed.

In some embodiments, when the secondary device 210 is an RF perforation device, the proximal portion 212 of the perforation device 210 may include an electrically insulated outer surface. As such, in certain embodiments, the proximal portion 212 can be handled directly by the user when the perforation device 210 is energized. In some embodiments, the proximal portion 212 is of a unitary construction formed entirely of an electrically insulative material. In certain embodiments, one exemplary class of materials for construction of the proximal portion 212 can include various grades of polytetrafluoroethylene (PTFE), polyetheretherketone (PEEK), among others. In some embodiments, the proximal portion 212 can further include reinforcing elements, e.g., a polymeric braid or coil, to enhance the structural properties, e.g., stiffness, torque transfer capability, and the like. In some embodiments, the proximal portion 212 is formed of a metal (e.g., a metal hypotube), and includes an outer electrically insulating layer.

In certain embodiments, the distal portion 214 is electrically conductive and is capable of transferring radiofrequency energy supplied by an external RF generator to the functional tip 216 for subsequent delivery to the target tissue in a transseptal crossing or an epicardial and/or endocardial ablation procedure, as described above. Any biocompatible electrically conductive material may be selected for construction of the distal portion 214. Exemplary materials may include stainless steel, nickel-titanium alloy, and the like. Further, for ease of illustration, the distal portion 214 is depicted in FIG. 2 as a single solid structure, although the construction of the distal portion 214 can vary to accommodate the particular structural requirements for the perforation device 210, as will be further explained below. For example, in some embodiments, the distal portion 214 can be constructed as a solid rod, a tube or a coil.

Additionally, in certain embodiments, the distal portion 214 can be constructed in multiple segments, e.g., a solid rod or hypotube in the regions nearest the proximal portion 212, and a coiled structure more distally to provide enhanced flexibility and torqueability. In some embodiments, the distal portion 214 can have a composite construction, e.g., a solid or tubular core conductor surrounded by a wire coil. Additionally, as shown in the illustrated embodiment, the proximal and distal portions 212, 214 are substantially isodiametric, although this is not a strict requirement in all embodiments.

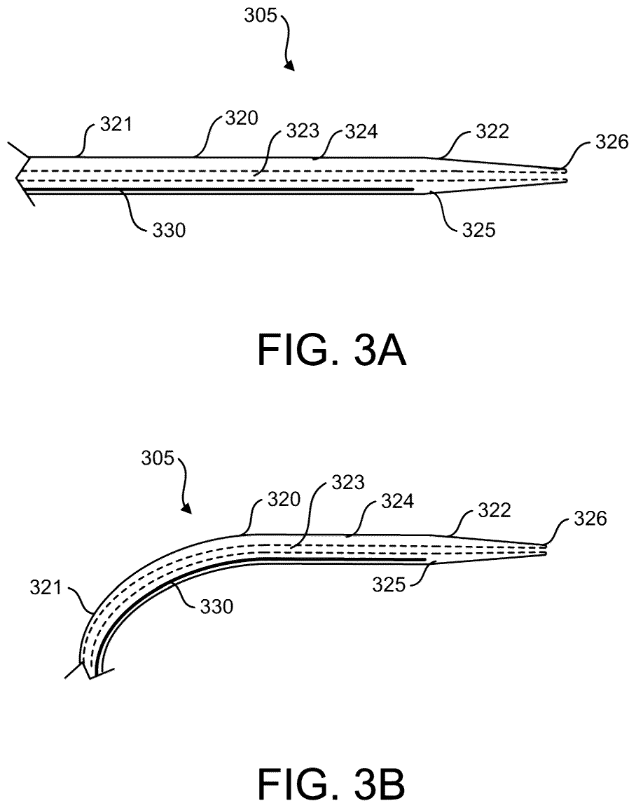

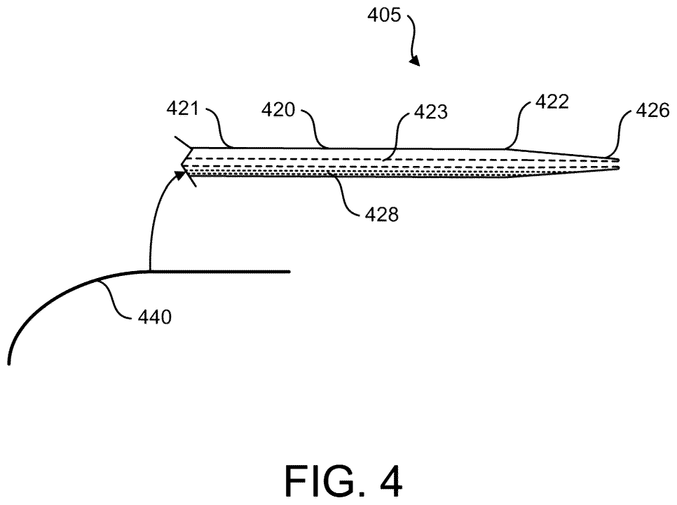

FIGS. 3A-4 illustrate a system for perforating a tissue within a heart having a delivery device 305, 405 and a shapeable pre-formed member 330, 440 which provides flexibility and patency to the delivery device 305, 405, according to an embodiment of the present disclosure. In an embodiment, the delivery device 305, 405 may be substantially structurally and functionally identical to the delivery device 205 of FIG. 2, except as described in connection with FIGS. 3A-4. As shown, the delivery device 305, 405 includes an elongated body 320, 420 having a body length, a proximal end portion 321, 421 and an opposite distal end portion 322, 422 terminating in a distal tip 326, 426. As shown, the delivery device 305, 405 further includes the elongated body 320, 420 defining a delivery lumen 323, 423 extending through the elongate body 320, 420 and dimensioned to slidingly receive at least one or more wires. In an embodiment, as discussed above, the one or more wires may include a secondary device. In an embodiment, the second device may include a perforation device, a guidewire, among others. Additionally, the system for perforating a tissue further includes a shapeable pre-formed member 330, 440 longitudinally advanced through the elongated body 320, 420 of the delivery device 305, 405. As shown, the shapeable pre-formed member 330, 440 does not extend longitudinally within the delivery lumen 323, 423 of the delivery device 305. Thus, the shapeable pre-formed member 330, 440 may be inserted within the device 305, 405 separate from the functional inner delivery. In an embodiment, as will be discussed further below, the pre-formed member 330, 440 maintains lumen patency of the delivery device 305, 405.

In an embodiment, as shown in FIG. 4, the delivery device may further include at least one or more support lumens 428. In an embodiment, the at least one or more support lumens 428 may be adjacent the delivery lumen 423. In certain embodiments, the pre-formed member 440 is longitudinally advanced through the at least one support lumens 428 of the delivery device 405. In an embodiment, the pre-formed member 330, 440 may be conformed into a desired shape by a user before being placed within the elongate body 320, 420 of the device 305, 405. In certain embodiments, the pre-formed member 440 may be longitudinally advanced through the at least one support lumens 428 of the delivery device 405 to achieve a specific device shape as the delivery device 405 is advancing within the heart.

In yet other embodiments, as shown in FIGS. 3A-3B, the elongate body 320 of the delivery device 305 may further include a device wall 325, 324 adjacent to the delivery lumen 323. In an embodiment, as shown, the pre-formed member 330 may be longitudinally advanced through a portion of the wall 325 of the elongated body 320 of the device 305. In certain embodiments, the pre-formed member 330 is embedded into the wall 325, 324 of the delivery device 305. Thus, in some embodiments, the pre-formed member 330 does not make contact or is not exposed to the delivery lumen 323. In an embodiment, the pre-formed member 330 is embedded into the wall 325, 324 of the delivery device 305 by any suitable method such as butt welding or use of adhesives. In an embodiment, the pre-formed member 330 may be longitudinally advanced through a portion of the upper wall 324 of the elongated body 320 of the device 305, and not the lower wall 325 of the device 305. In yet another embodiment, the pre-formed member 330 may be longitudinally advanced through the lower wall 325 or upper wall 324 and the other wall not containing the pre-formed member 330 may include a secondary lumen used for other purposes.

In some embodiments, the pre-formed member 330, 440 allows for flexibility to the device while minimizing kinking of the elongated body 320, 420 of the device 305, 405. In an embodiment, the pre-formed member 330, 440 may shape the elongate body 320, 420 of the device 305, 405. In certain embodiments, the pre-formed member 330, 440 may be used to add stiffness to the elongate body 320, 420 of the delivery device. In an embodiment, as the delivery device 305, 405 is navigated within the body and specifically the heart of a patient, the shape of the device, along with the inner diameter of the delivery lumen 323, 423, may vary. In some embodiments, this may include some kinking of the elongate body 320, 420 and the delivery lumen 323, 423. In an embodiment, the pre-formed member 330, 440 allows for less kinking of the delivery device 305, 405, maintaining a consistent inner diameter of the delivery lumen 323, 423, and assures that the delivery lumen 323, 423 is not obstructed by the kinking of the device and is usable to cannulate one or more other devices or substances through. Thus, in this way, the pre-formed member 330, 440 enhances the flexibility and durability of the delivery device by minimizing the risk of kinking and maintaining optimal lumen patency.

In an embodiment, the pre-formed member 330, 440 is solid, i.e., a mandrel, and does not define its own lumen. In some embodiments, the pre-formed member 330, 340 may be made of material having the characteristic of shape memory. In an embodiment, the pre-formed member is made of nitinol, stainless steel and a combination thereof. In some embodiments, the pre-formed member 330, 440 may be longitudinally advanced through the entire elongated body 320, 420 of the delivery device. In certain embodiments, the pre-formed member 330, 440 may be longitudinally advanced through a portion of the elongate body 320, 420 of the delivery device 305, 405 in a portion in which adjustability or a desired stiffness is required.

In an embodiment, the delivery device 305, 405 is a dilator comprising an elongated body having a body length, a proximal end and a distal end portion terminating in a distal tip, the elongated body defining a delivery lumen extending through the elongate body and dimensioned to slidingly receive at least one or more wires. In certain embodiments, the dilator may include at least one support lumens, and the pre-formed member is longitudinally advanced through the at least one support lumens of the dilator. In some embodiments, the delivery device 305, 405 is an elongated catheter comprising an elongated body having a body length, a proximal end and a distal end portion terminating in a distal tip, the elongated body defining a delivery lumen extending through the elongate body and dimensioned to slidingly receive at least one or more devices. In an embodiment, the elongated catheter is adapted to receive and allow longitudinal translation of the pre-formed member therein.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for perforating a tissue within a heart, the system comprising:
a delivery device comprising an elongated body having a body length, a proximal end and a distal end portion terminating in a distal tip, the elongated body defining a delivery lumen extending through the elongate body and dimensioned to slidingly receive at least one or more wires;
a secondary device having a proximal end and a distal end portion terminating in a distal tip dimensioned to extend longitudinally within the delivery lumen of the delivery device towards the distal tip of the device; and
a shapeable pre-formed member longitudinally advanced through the elongated body of the delivery device, wherein the shapeable pre-formed member does not extend longitudinally within the delivery lumen of the delivery device, and wherein the pre-formed member maintains lumen patency of the delivery device.

2. The system of claim 1, wherein the delivery device further includes at least one support lumens, wherein the at least one support lumens is adjacent the delivery lumen, and wherein the pre-formed member is longitudinally advanced through the at least one support lumens.

3. The system of claim 1, wherein the elongated body of the delivery device further includes a device wall adjacent to the delivery lumen, and wherein the pre-formed member is longitudinally advanced through a portion of the wall of the elongated body.

4. The system of claim 3, wherein the pre-formed member is embedded into the wall of the delivery device.

5. The system of claim 1, wherein the pre-formed member allows flexibility to the device while minimizing kinking of the elongated body of the device.

6. The system of claim 1, wherein the pre-formed member is conformed into a desired shape by a user.

7. The system of claim 6, wherein the pre-formed member is longitudinally advanced through the at least one support lumens of the delivery device to achieve a specific device shape as the delivery device is advancing within the heart.

8. The system of claim 1, wherein the delivery device is a dilator comprising at least one support lumens, and wherein the pre-formed member is longitudinally advanced through the at least one support lumens of the dilator.

9. The system of claim 1, wherein the delivery device is an elongated catheter, and wherein the elongated catheter is adapted to receive and allow longitudinal translation of the pre-formed member therein.

10. The system of claim 1, wherein the pre-formed member is solid, and wherein the pre-formed member does not define a lumen.

11. The system of claim 1, wherein the pre-formed member is selected from the group consisting of stainless steel and nitinol.

12. A system for perforating a tissue within a heart, the system comprising:
a delivery device comprising an elongated body having a body length, a proximal end and a distal end portion terminating in a distal tip, the elongated body defining a delivery lumen and at least one support lumens extending through the elongate body and dimensioned to slidingly receive at least one or more wires;
a puncture device having a proximal end and a distal end portion terminating in a distal tip dimensioned to extend longitudinally within the delivery lumen of the delivery device towards the distal tip of the device; and
a shapeable pre-formed member longitudinally advanced through the at least one support lumens of the delivery device, wherein the pre-formed member maintains lumen patency of the delivery device.

13. The system of claim 12, wherein the pre-formed member is longitudinally advanced through the at least one support lumens of the delivery device to achieve a specific device shape as the delivery device is advancing within the heart.

14. The system of claim 12, wherein the delivery device is a dilator comprising at least one support lumens, and wherein the pre-formed member is longitudinally advanced through the at least one support lumens of the dilator.

15. The system of claim 12, wherein the delivery device is an elongated catheter, and wherein the elongated catheter is adapted to receive and allow longitudinal translation of the pre-formed member therein.

16. The system of claim 12, wherein the pre-formed member is solid, and wherein the pre-formed member does not define a lumen.

17. The system of claim 12, wherein the pre-formed member is selected from the group consisting of stainless steel and nitinol.

18. The system of claim 12, wherein the pre-formed member is longitudinally advanced through the entire elongated body of the delivery device.

19. The system of claim 12, wherein the pre-formed member is longitudinally advanced through a portion of the elongated body of the delivery device.

20. A method for perforating a tissue within a heart, the method comprising:
providing a delivery device comprising an elongated body having a body length, a proximal end and a distal end portion terminating in a distal tip, the elongated body defining a delivery lumen extending through the elongate body and dimensioned to slidingly receive at least one or more wires;
advancing a secondary device having a proximal end and a distal end portion terminating in a distal tip dimensioned to extend longitudinally within the delivery lumen of the delivery device towards the distal tip of the device; and
advancing a shapeable pre-formed member longitudinally advanced through the elongated body of the delivery device, wherein the shapeable pre-formed member does not extend longitudinally within the delivery lumen of the delivery device, and wherein the pre-formed member maintains lumen patency of the delivery device.
